(19) United States
(12) Patent Application Publication
(10) Pub. No.: US 2008/0234673 A1
(43) Pub. Date: Sep. 25, 2008

(54) MULTI-ELECTRODE INSTRUMENTS

(75) Inventors: Duane W. Marion, Santa Clara, CA (US); John J. Lorton, Campbell, CA (US)

Correspondence Address: ARTHROCARE CORPORATION, 7500 Rialto Boulevard, Building Two, Suite 100, Austin, TX, 78735-8532, US

(73) Assignee: ArthroCare Corporation, Sunnyvale, CA (US)

(21) Appl. No.: 11/688,398

(22) Filed: Mar. 20, 2007

(51) Int. Cl. A61B 18/14

(52) U.S. Cl. 606/45; 606/41

(57) ABSTRACT

Multi-electrode instruments and methods for applying electrical energy to the multiple electrodes are described. An assembly having at least two electrodes may be configured such that the electrodes are positioned at an angle relative to one another and/or are each configured to treat different tissue types. Moreover, power may be applied to one, some, or all of the electrodes utilizing various switching systems and/or electrical monitoring systems.

### FIELD OF THE INVENTION

The present invention relates to electrosurgical instruments having multiple electrodes. More particularly, the present invention relates to electrosurgical instruments having multiple electrodes in various configurations which allow treatment of different tissue types with a single instrument.

### BACKGROUND OF THE INVENTION

Conventional electrosurgical methods generally reduce patient bleeding associated with tissue cutting operations and improve the surgeon's visibility. These electrosurgical devices and procedures, however, suffer from a number of disadvantages. For example, monopolar and/or bipolar electrosurgical devices are typically designed for treating certain tissue types. One specific electrosurgical device may be effective for ablating a first tissue type such as cartilage, yet ineffective for treating a second tissue type, such as loose or elastic connective tissue like the synovial tissue in joints.

Likewise, during certain electrosurgical procedures such as the removal or resection of the meniscus during arthroscopic surgery to the knee, it is generally necessary to employ two different tissue removal devices, namely an arthroscopic punch and a shaver. The use of multiple instruments brings with it the associated problems not only with preparation and cost but also with the insertion and removal of multiple instruments from the patient body. There is a need for an electrosurgical instrument which enables the treatment of more than one tissue type, such as for the removal of fibrocartilaginous tissue as well as softer tissue. Moreover, there is a need for the same device which is adapted for aspirating resected tissue, excess fluids, and ablation by-products from the surgical site.

Electrosurgical instruments which can treat multiple tissue types may utilize multiple electrodes, however, splitting power from a power supply between different types of active electrodes may be problematic with respect to heating of the instrument and tissue as well as with power consumption. Accordingly, there is also a need for methods and apparatus to control the power delivery of such instruments which utilize multiple electrodes.

### SUMMARY OF THE INVENTION

A single electrosurgical instrument having multiple electrodes in various configurations may be used to treat more than one type of tissue, thereby eliminating the need for multiple instruments or for inserting and removing more than a single instrument into a treatment space within a patient body. Accordingly, such a single instrument may: (1) volumetrically remove tissue, bone or cartilage (i.e., ablate or effect molecular dissociation of the tissue structure); (2) cut or resect tissue; (3) shrink or contract collagen connective tissue; and/or (4) coagulate severed blood vessels.

High electric field intensities may be generated by applying a high frequency voltage that is sufficient to vaporize an electrically conductive fluid over at least a portion of the active electrode(s) in the region between the distal tip of the active electrode(s) and the target tissue. The electrically conductive fluid may be a gas or liquid, such as isotonic saline, delivered to the target site, or a viscous fluid, such as a gel, that is located at the target site. In the latter embodiment, the active electrode(s) are submersed in the electrically conductive gel during the surgical procedure. Since the vapor layer or vaporized region has relatively high electrical impedance, it minimizes the current flow into the electrically conductive fluid. This ionization, under optimal conditions, induces the discharge of energetic electrons and photons from the vapor layer to the surface of the target tissue. A more detailed description of this phenomenon, termed Coblation®, can be found in commonly assigned U.S. Pat. No. 5,697,882 the complete disclosure of which is incorporated herein by reference in its entirety.

In utilizing such an electrode assembly having at least a first electrode and a second electrode, each respective electrode may be individually powered by a common or separate power supply and they may each have their own respective return electrode or share a common return electrode. Independently powered electrodes or electrodes sharing a common power supply may be utilized.

Each respective active electrode and the return electrode may be insulated via an insulating material such as a ceramic or other insulating material such as polytetrafluoroethylene, polyimide, etc. Additionally, one or more lumen openings may be defined along the electrode assembly for infusing, injecting, drawing or suctioning fluid and debris from the ablation site and through the shaft for removal from the body.

Examples of a multi-electrode assembly may utilize a first electrode which forms an interdigitating member that projects between members of a second electrode with an insulating material separating the electrodes. Alternatively, the electrodes may be positioned adjacent to one another along a common surface. In additional variations, the electrode assembly may utilize a first electrode positioned at an angle, e.g., 90°, relative to a longitudinal axis of the shaft. A second electrode may be positioned at a distal end of the assembly such that first and second electrodes are separated and angled relative to one another.

One or both electrodes may be configured into various configurations to effect treatments such as tissue ablation, cutting, or resection. Additionally, one or both electrodes may include a fluid lumen for infusing a fluid such as saline and/or for drawing debris and fluid back into the openings. Both electrodes may be electrically isolated from one another as well as from a common return electrode by an insulator. Such an assembly utilizing multiple electrodes in different configurations may allow the user to utilize a single device for treating different tissue regions within, e.g., a joint, where space is limited without having to withdraw and introduce multiple instruments into the tissue region.

In utilizing the two or more active electrodes on a single electrosurgical instrument in any of variations described herein, a relay or switch may be used to select which of the electrodes are powered to deliver the output energy. Such a switch may be actuated manually by the user or automatically by a controller. With each electrode being electrically isolated from one another and from the return electrode, the current flowing through the electrode assembly is applied to the tissue to be treated. Each electrode may be configured into any of the variations described herein or as known in the art and in any combination of different electrode types on a single instrument to effect the treatment of multiple tissue types utilizing a single electrosurgical device.

In yet additional variations where an electrode assembly has more than two electrodes, each electrically isolated electrode may each include an individually actuatable relay. The electrodes may be connected in parallel with one another and with a common return electrode. Each of the relays may be individually actuatable such that the current may be applied to one, all, or any combination of the electrodes to effect the desired tissue treatment.

Each of the isolated electrodes may be designed such that each includes a voltage and/or current measurement device to measure each applied parameter. Such a configuration may be applied to all or a few of the electrodes utilized. With these measured values, impedance and power loads may be calculated. Once an ablative effect has been established at one particular electrode upon the tissue being treated, the load impedance generally increases. With changes in the load impedance detected, a generator control circuitry, e.g., a microprocessor or hardware controller, may be configured to track changes in the load impedance at a given electrode and to make a determination to activate subsequent electrodes.

As the tissue is treated, the voltage meter and ammeter may monitor their respective signals which are used to calculate load impedance. When the load impedance reaches a predetermined threshold level, the system may be configured to then actuate relay to activate the electrode. This process may be repeated until all relays have been actuated and all electrodes are activated. Alternatively, the processor may be configured to activate subsequent electrodes based upon the measured current or the delivered power to minimize any current or power spikes initially delivered to the electrodes to facilitate the ablative effects on the tissue being treated.

With the potential of activating multiple electrodes, one method for limiting the power that can be delivered to each electrode is to limit activation of a particular electrode during a power cycle. Each active electrodes may be electrically connected to the power supply through respective diodes. When the power supply is activated, the respective diodes may limit the activation of each electrode to only half of each cycle of the output waveform (or to 1/N of each cycle of the output waveform, where N is the number of active electrodes through which current is flowing). Use of the diodes may help to ensure that the power is equally shared between each active electrode independently of the load that may exist between each electrode and the return electrode.

While a single power supply may be shared between multiple numbers of electrodes, another variation is to power each electrode from an independent, separately controlled power supply. Each power supply can be independently adjusted depending upon the measured current levels received from each electrode assembly to maintain a constant level of power applied by the multiple electrodes at the tissue site.

### BRIEF DESCRIPTION OF THE DRAWINGS

FIG. 1 shows an exemplary electrosurgical system for a single instrument having multiple electrodes configured to treat varying tissue regions.

FIG. 2 illustrates an exemplary electrosurgical probe which generally includes an elongated shaft which may be flexible or rigid, a handle coupled to the proximal end of shaft and a multi-electrode assembly.

FIG. 3 illustrates a perspective view of one variation where the electrode assembly may have at least a first electrode and a second electrode positioned proximally thereof.

FIG. 4 shows another variation of a multi-electrode assembly disposed upon the shaft and having first and second active electrodes positioned at an angle relative to a longitudinal axis of the shaft.

FIG. 5 shows an end view of an alternative example of a multi-electrode assembly having a first electrode which forms an interdigitating member that projects between members of the second electrode.

FIG. 6 shows an end view of another example of a multi-electrode assembly similar to FIG. 5.

FIG. 7 shows another electrode assembly configuration where first and second electrodes are configured into wedge-shaped electrodes which are placed in apposition to one another.

FIG. 8 shows another variation where the first and second electrodes may each include an arcuate extension which curves circumferentially with respect to the assembly.

FIG. 9 shows another variation similar to that in FIG. 8 where the electrode assembly has a non-circular cross-sectional profile.

FIG. 10A shows a perspective view of an electrode assembly which utilizes a circumferentially-shaped first electrode which at least partially surrounds a second electrode.

FIGS. 10B and 10C show perspective side and end views, respectively, of the assembly of FIG. 10A.

FIG. 11A shows a perspective side view of an electrode assembly having its first and second electrodes separated and positioned at an angle from one another.

FIG. 11B shows a perspective side view of another variation of an electrode assembly having various electrode configurations.

FIG. 11C shows a perspective side view of another variation of an electrode assembly having additional electrode configurations.

FIGS. 12A and 12B schematically illustrate variations for switching between multiple electrodes in a single electrosurgical instrument.

FIG. 13 schematically illustrates an electrode assembly having four electrodes each being individually actuatable with a common return electrode.

FIG. 14 illustrates an example of a voltage meter connected in parallel with a power source and/or ammeter connected in series with a particular electrode to measure the applied voltage and current, respectively.

FIG. 15 illustrates another variation of an electrode assembly utilizing multiple electrodes in an electrode assembly.

FIG. 16 illustrates one example for limiting the power that can be delivered to each electrode when multiple electrodes are activated.

FIGS. 17A and 17B illustrate examples for variations on delivering power to multiple electrodes from independent, separately controlled power supplies.

### DETAILED DESCRIPTION OF THE INVENTION

High frequency (RF) electrical energy may be applied to one or more active electrodes in the presence of electrically conductive fluid to remove and/or modify the structure of tissue structures. Depending on the specific procedure, a single instrument having multiple electrodes in various configurations may be used to: (1) volumetrically remove tissue, bone or cartilage (i.e., ablate or effect molecular dissociation of the tissue structure); (2) cut or resect tissue; (3) shrink or contract collagen connective tissue; and/or (4) coagulate severed blood vessels.

In these procedures, a high frequency voltage difference is applied between the active electrode(s) and one or more return electrode(s) to develop high electric field intensities in the vicinity of the target tissue site. The high electric field intensities lead to electric field induced molecular breakdown of target tissue through molecular dissociation (rather than thermal evaporation or carbonization). This molecular disintegration completely removes the tissue structure, as opposed to dehydrating the tissue material by the removal of liquid from within the cells of the tissue, as is typically the case with electrosurgical desiccation and vaporization.

The high electric field intensities may be generated by applying a high frequency voltage that is sufficient to vaporize an electrically conductive fluid over at least a portion of the active electrode(s) in the region between the distal tip of the active electrode(s) and the target tissue. The electrically conductive fluid may be a gas or liquid, such as isotonic saline, delivered to the target site, or a viscous fluid, such as a gel, that is located at the target site. In the latter embodiment, the active electrode(s) are submersed in the electrically conductive gel during the surgical procedure. Since the vapor layer or vaporized region has relatively high electrical impedance, it minimizes the current flow into the electrically conductive fluid. This ionization, under optimal conditions, induces the discharge of energetic electrons and photons from the vapor layer to the surface of the target tissue. A more detailed description of this phenomenon, termed Coblation®, can be found in commonly assigned U.S. Pat. No. 5,683,366 the complete disclosure of which is incorporated herein by reference in its entirety.

A plasma may be generated in the vicinity of the active electrode on application of the voltage to the electrodes in the presence of the electrically conductive fluid. The plasma includes energetic electrons, ions, photons and the like that are discharged from a vapor layer of the conductive fluid, as described in greater detail in U.S. Pat. No. 5,697,882 the complete disclosure which is incorporated herein by reference in its entirety.

The systems and methods for selectively applying electrical energy to a target location within or on a patient's body may be accomplished particularly in procedures where the tissue site is flooded or submerged with an electrically conductive fluid, such as during arthroscopic surgery of the knee, shoulder, ankle, hip, elbow, hand, foot, etc. Other tissue regions which may be treated by the system and methods described herein may also include, but are not limited to, prostate tissue, and leiomyomas (fibroids) located within the uterus, gingival tissues and mucosal tissues located in the mouth, tumors, scar tissue, myocardial tissue, collagenous tissue within the eye or epidermal and dermal tissues on the surface of the skin, etc. Other procedures which may be performed may also include laminectomy/disectomy procedures for treating herniated disks, decompressive laminectomy for stenosis in the lumbosacral and cervical spine, posterior lumbosacral and cervical spine fusions, treatment of scoliosis associated with vertebral disease, foraminotomies to remove the roof of the intervertebral foramina to relieve nerve root compression, as well as anterior cervical and lumbar disectomies. Tissue resection within accessible sites of the body that are suitable for electrode loop resection, such as the resection of prostate tissue, leiomyomas (fibroids) located within the uterus, and other diseased tissue within the body, may also be performed

Other procedures which may be performed where multiple tissue types are present may also include, e.g., the resection and/or ablation of the meniscus and the synovial tissue within a joint during an arthroscopic procedure. It will be appreciated that the systems and methods described herein can be applied equally well to procedures involving other tissues of the body, as well as to other procedures including open procedures, intravascular procedures, urology, laparoscopy, arthroscopy, thoracoscopy or other cardiac procedures, dermatology, orthopedics, gynecology, otorhinolaryngology, spinal and neurologic procedures, oncology, and the like.

The electrosurgical instrument may comprise a shaft or a handpiece having a proximal end and a distal end which supports the one or more active electrodes. The shaft or handpiece may assume a wide variety of configurations, with the primary purpose being to mechanically support the active electrode and permit the treating physician to manipulate the electrodes from a proximal end of the shaft. The shaft may be rigid or flexible, with flexible shafts optionally being combined with a generally rigid external tube for mechanical support. The distal portion of the shaft may comprise a flexible material, such as plastics, malleable stainless steel, etc, so that the physician can mold the distal portion into different configurations for different applications. Flexible shafts may be combined with pull wires, shape memory actuators, and other known mechanisms for effecting selective deflection of the distal end of the shaft to facilitate positioning of the electrode array. The shaft will usually include a plurality of wires or other conductive elements running axially therethrough to permit connection of the electrode array to a connector at the proximal end of the shaft. Thus, the shaft may typically have a length between at least 5 cm and at least 10 cm, more typically being 20 cm or longer for endoscopic procedures. The shaft may typically have a diameter of at least 0.5 mm and frequently in the range of from about 1 mm to 10 mm. Of course, in various procedures, the shaft may have any suitable length and diameter that would facilitate handling by the surgeon.

As mentioned above, a gas or fluid is typically applied to the target tissue region and in some procedures it may also be desirable to retrieve or aspirate the electrically conductive fluid after it has been directed to the target site. In addition, it may be desirable to aspirate small pieces of tissue that are not completely disintegrated by the high frequency energy, air bubbles, or other fluids at the target site, such as blood, mucus, the gaseous products of ablation, etc. Accordingly, the instruments described herein can include a suction lumen in the probe or on another instrument for aspirating fluids from the target site.

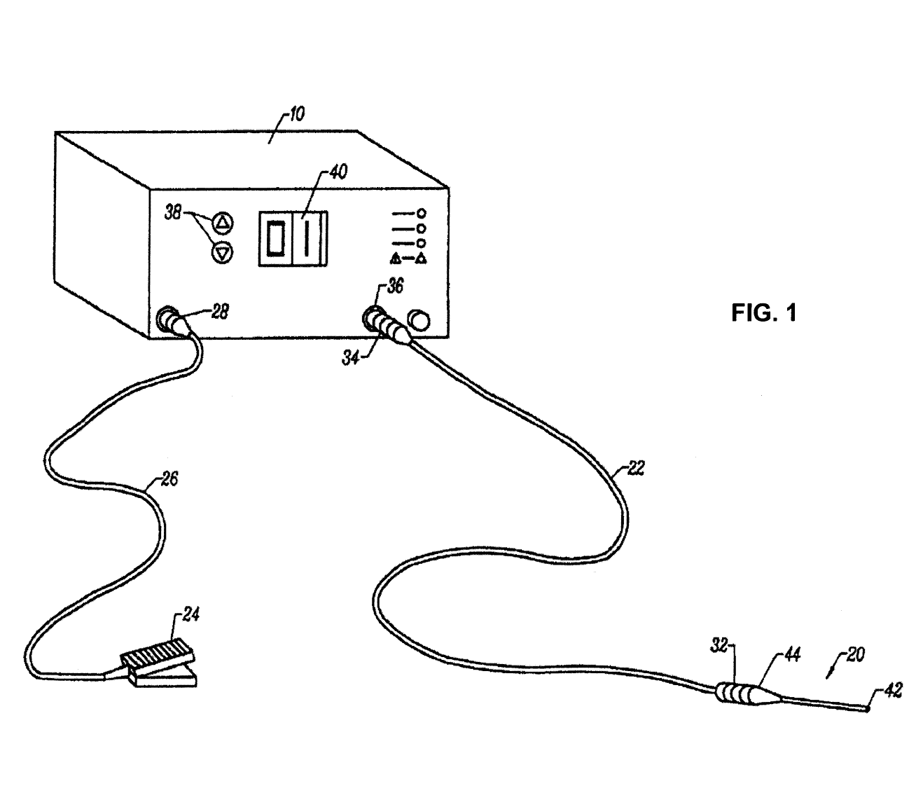

Referring to FIG. 1, an exemplary electrosurgical system for a single instrument having multiple electrodes configured to treat varying tissue regions is illustrated in the assembly. As shown, the electrosurgical system may generally comprise an electrosurgical probe 20 connected to a power supply 10 for providing high frequency voltage to the active electrodes. Probe 20 includes a connector housing 44 at its proximal end, which can be removably connected to a probe receptacle 32 of a probe cable 22. The proximal portion of cable 22 has a connector 34 to couple probe 20 to power supply 10 to power the multiple electrodes of electrode assembly 42 positioned near or at the distal end of probe 20.

Power supply 10 has an operator controllable voltage level adjustment 38 to change the applied voltage level, which is observable at a voltage level display 40. Power supply 10 may also include one or more foot pedals 24 and a cable 26 which is removably coupled to a receptacle with a cable connector 28. The foot pedal 24 may also include a second pedal (not shown) for remotely adjusting the energy level applied to the active electrodes and a third pedal (also not shown) for switching between an ablation mode and a coagulation mode or for switching to activate between electrodes. Operation of and configurations for the power supply 10 are described in further detail in U.S. Pat. No. 6,746,447, which is incorporated herein by reference in its entirety.

The voltage applied between the return electrodes and the active electrodes may be at high or radio frequency, typically between about 5 kHz and 20 MHz, usually being between about 30 kHz and 2.5 MHz, preferably being between about 50 kHz and 500 kHz, more preferably less than 350 kHz, and most preferably between about 100 kHz and 200 kHz. The RMS (root mean square) voltage applied will usually be in the range from about 5 volts to 1000 volts, preferably being in the range from about 10 volts to 500 volts depending on the active electrode size, the operating frequency and the operation mode of the particular procedure or desired effect on the tissue (i.e., contraction, coagulation or ablation). Typically, the peak-to-peak voltage will be in the range of 10 to 2000 volts, preferably in the range of 20 to 1200 volts and more preferably in the range of about 40 to 800 volts (again, depending on the electrode size, the operating frequency and the operation mode).

The power source may be current limited or otherwise controlled so that undesired heating of the target tissue or surrounding (non-target) tissue does not occur. In one variation, current limiting inductors are placed in series with each independent active electrode, where the inductance of the inductor is in the range of 10 uH to 50,000 uH, depending on the electrical properties of the target tissue, the desired tissue heating rate and the operating frequency. Alternatively, capacitor-inductor (LC) circuit structures may be employed, as described previously in PCT application WO 94/026228, which is incorporated herein by reference in its entirety.

Additionally, current limiting resistors may be selected. These resistors will have a large positive temperature coefficient of resistance so that, as the current level begins to rise for any individual active electrode in contact with a low resistance medium (e.g., saline irrigant or conductive gel), the resistance of the current limiting resistor increases significantly, thereby minimizing the power delivery from the active electrode into the low resistance medium (e.g., saline irrigant or conductive gel).

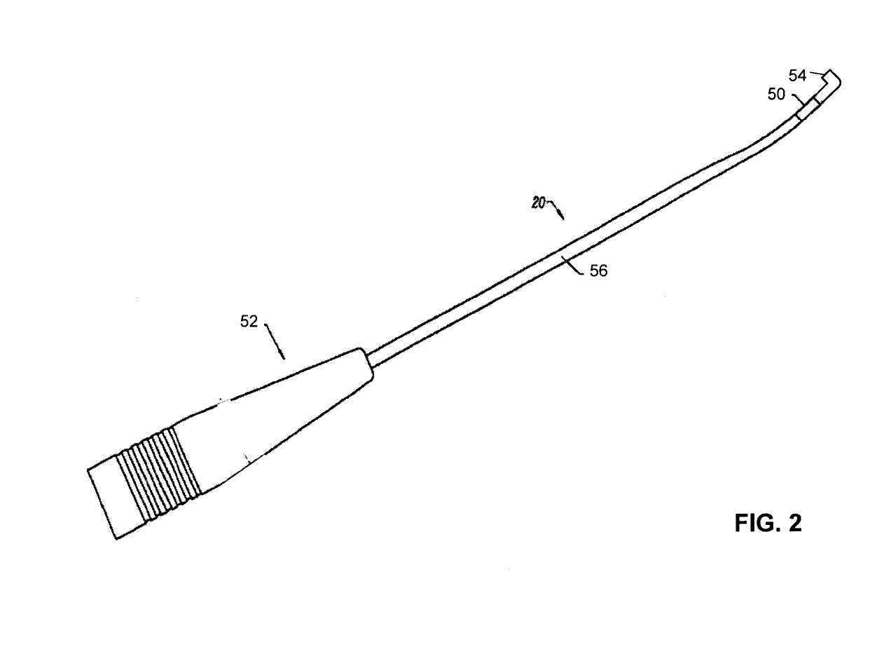

FIG. 2 illustrates an exemplary electrosurgical probe 20 which generally includes an elongated shaft 50 which may be flexible or rigid, a handle 52 coupled to the proximal end of shaft 50 and a multi-electrode assembly 54, described in further detail below, coupled to the distal end of shaft 50. Shaft 50 may comprise an electrically conducting material, such as metal, which may be selected from the group consisting of, e.g., tungsten, stainless steel alloys, platinum or its alloys, titanium or its alloys, molybdenum or its alloys, and nickel or its alloys. Shaft 50 also includes an electrically insulating jacket, which is typically formed as one or more electrically insulating sheaths or coatings, such as polytetrafluoroethylene, polyimide, and the like. The provision of the electrically insulating jacket over the shaft prevents direct electrical contact between these metal elements and any adjacent body structure or the surgeon. Such direct electrical contact between a body structure (e.g., tendon) and an exposed electrode could result in unwanted heating of the structure at the point of contact causing necrosis.

Handle 52 typically comprises a plastic material that is easily molded into a suitable shape for handling by the surgeon. Moreover, the distal portion of shaft 50 may be bent to improve access to the operative site of the tissue being treated (e.g., contracted). In alternative embodiments, the distal portion of shaft 50 comprises a flexible material which can be deflected relative to the longitudinal axis of the shaft. Such deflection may be selectively induced by mechanical tension of a pull wire, for example, or by a shape memory wire that expands or contracts by externally applied temperature changes. A more complete description of this embodiment can be found in PCT application WO 94/026228, which has been incorporated by reference above.

The bend in the distal portion of shaft 50 is particularly advantageous in arthroscopic treatment of joint tissue as it allows the surgeon to reach the target tissue within the joint as the shaft 50 extends through a cannula or portal. Of course, it will be recognized that the shaft may have different angles depending on the procedure. For example, a shaft having a 90° bend angle may be particularly useful for accessing tissue located in the back portion of a joint compartment and a shaft having a 10° to 30° bend angle may be useful for accessing tissue near or in the front portion of the joint compartment.

Regardless of the bend angle, an electrode assembly having multiple, e.g., two or more, actuatable electrodes disposed near or at the distal end of shaft 50 may be utilized. General difficulties in designing electrosurgical devices with relatively large active electrodes typically entail delivering a relatively high level of RF energy until ablative effects are activated at the electrodes. However, once the ablative effects are activated, the load impedance increases and the power delivery to the tissue decreases. Thus, a multi-electrode assembly may be configured to effectively deliver the energy to a tissue region of interest.

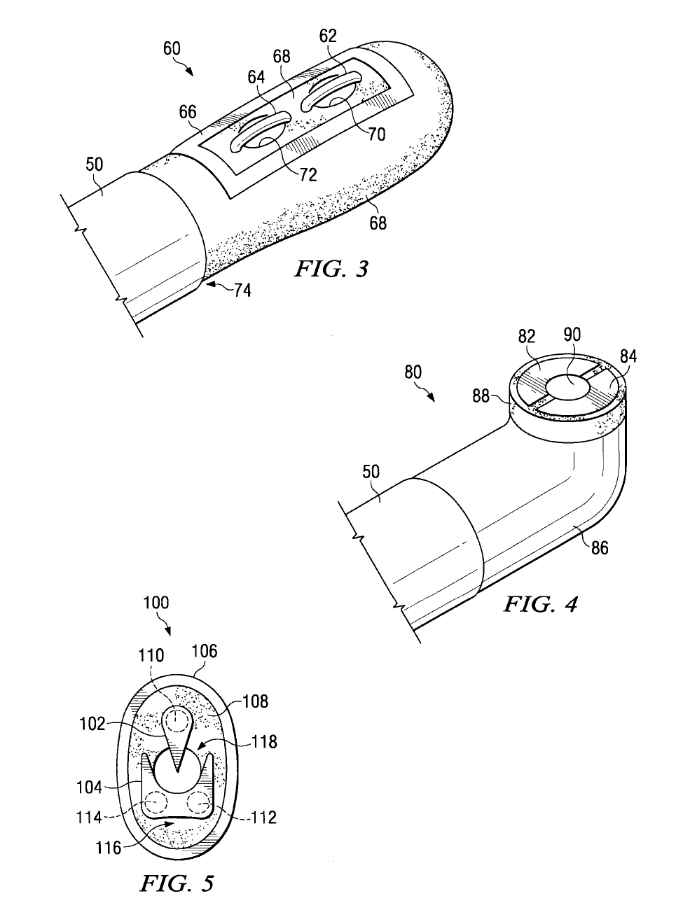

FIG. 3 illustrates a perspective view of one such variation where electrode assembly 60 may have at least a first electrode 62 and a second electrode 64 positioned proximally thereof. Each respective electrode 62, 64 may be individually powered by a common or separate power supply and they may each have the own respective return electrode or share a common return electrode 66, as illustrated in this example. Independently powered electrodes may improve the ablation performance of the electrode assembly because if the generated plasma field dissipated at one of the active electrodes, the system may be able to maintain the plasma field at least at the second electrode. In contrast, a single electrode device will deliver the majority of its RF current at the location of lowest impedance, which may not allow the system of maintain a higher impedance plasma field at another location. Variations for powering and/or controlling the activation of different electrodes are described below in further detail.

Each respective active electrode 62,64 and the return electrode 66 may be insulated via an insulating material 68 such as a ceramic or also as described above, such as polytetrafluoroethylene, polyimide, ceramic, etc. Additionally, one or more lumen openings, such as first opening 70 and/or second opening 72, may be defined along electrode assembly 60 for infusing, injecting, drawing or suctioning fluid and debris from the ablation site and through the shaft 50 for removal from the body. First and second openings 70, 72 may be separate or share a common fluid lumen and they may be defined over assembly 60, for example, adjacent to their respective active electrodes 62, 64. Additionally, a fluid such as saline may be delivered through shaft 50 to flood the tissue region to be treated. Thus, saline may be delivered through a flared opening 74 defined around shaft 50 proximally of electrode assembly 60.

The area of the tissue treatment surface of the electrodes can vary widely and the tissue treatment surface can assume a variety of geometries, with particular areas and geometries being selected for specific applications. Active electrode surfaces can have areas in the range, e.g., from 0.25 mm² to 75 mm², usually being from about 0.5 mm² to 40 mm². The geometries can be planar, concave, convex, hemispherical, conical, linear “in-line” array or virtually any other regular or irregular shape. Most commonly, the active electrode(s) or active electrode(s) will be formed at the distal tip of the electrosurgical probe shaft, frequently being planar, disk-shaped, or hemispherical surfaces for use in reshaping procedures or being linear arrays for use in cutting. Alternatively or additionally, the active electrode(s) may be formed on lateral surfaces of the electrosurgical probe shaft (e.g., in the manner of a spatula), facilitating access to certain body structures in endoscopic procedures.

Another example is illustrated in the perspective view of FIG. 4 which shows another variation of a multi-electrode assembly 80 disposed upon shaft 50. In this variation, the first and second active electrodes 82, 84 may be positioned at an angle relative to a longitudinal axis of shaft 50 to facilitate access to various tissue regions. Alternatively, assembly 80 may be aligned with the longitudinal axis of shaft 50 such that the active electrodes are distally disposed relative to shaft 50. In either case, first and second active electrodes 82, 84 may be positioned adjacent to one another in a semi-circular configuration, in this example, surrounding a fluid lumen 90. Although each active electrode 82, 84 may have its own separate return electrode, they may share a common return electrode 86 positioned apart from the active electrodes 82, 84 by insulator 88.

An alternative example of a multi-electrode assembly is shown in the end view of configuration 100 of FIG. 5. As shown, first electrode 102 may be affixed to assembly 100 via support 110 such that first electrode 102 forms an interdigitating member that projects between members of second electrode 104, which may be affixed to assembly 100 via supports 112, 114. Although first electrode 102 may project between second electrode 104, they may be separated such that they are non-contacting. An insulating material 108 may separate the electrodes 102, 104 not only from one another, but also from a common return electrode 106 located proximally of electrodes 102, 104. Moreover, there may be a gap or a clearance 116 between second electrode 104 and insulator 108 to allow for the unobstructed flow of saline into the area or for the removal of debris and fluids into fluid lumen 118, which may be defined between first and second electrodes 102, 104.

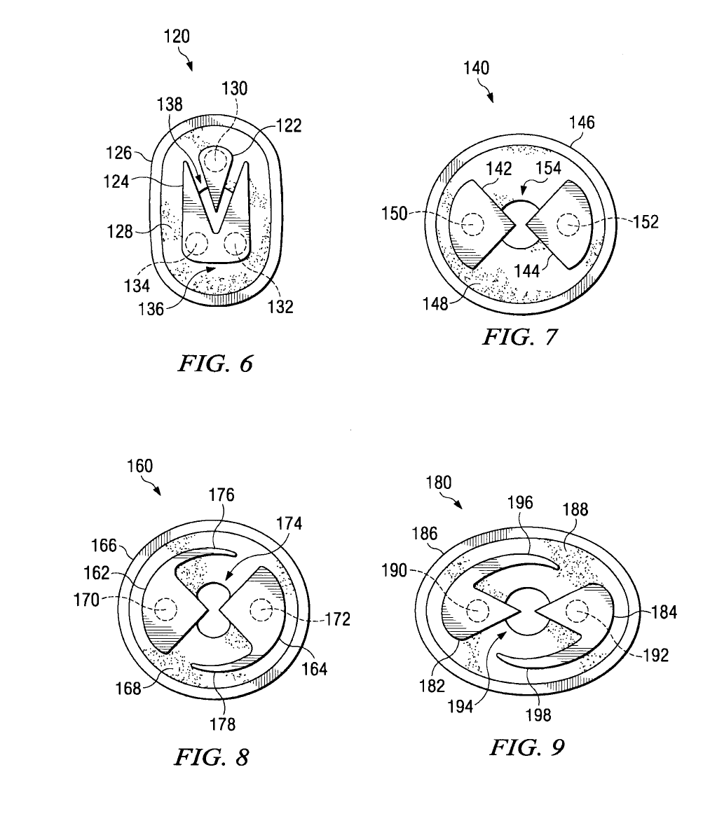

Another variation is illustrated in FIG. 6 where first electrode 122 affixed via support 130 to electrode assembly 120 and second electrode 124 affixed via supports 132, 134 to assembly 120 may be apposed to one another in an interdigitating configuration. Similarly, first and second electrodes 122, 124 may share a common return electrode 126 while separated via insulator 128. Moreover, gap or clearance 136 between second electrode 124 and insulator 128 may be defined to allow for fluid infusion and/or debris and fluid removal to lumen 138, defined between the active electrodes 122, 124. In this variation, first and second electrodes 122, 124 may define elongated members which interdigitate closely within one another relative to the ablation area of the assembly 120. Moreover, this variation as well as that illustrated in FIG. 5 may each define a cross-sectional area or shape similar to or approximating an elliptical configuration, as shown. Although illustrated in an elliptical shape, other configurations may be utilized, e.g., circles, triangular, hexagonal, etc.

FIG. 7 shows yet another electrode assembly configuration 140 where first and second electrodes 142, 144, each affixed to assembly 140 via supports 150, 152, respectively, may be configured into wedge-shaped electrodes which are placed in apposition to one another. Each wedge portion of these electrodes 142, 144 may form an angle of 90° with respect to the longitudinal axis of the assembly 140. Common return electrode 146 may be positioned proximally of the electrodes 142, 144 and they may each be separated by insulator 148. Fluid lumen opening 154 may also be seen defined between the electrodes 142, 144. In this variation, the assembly 140 may form a circular configuration, although other shapes may be utilized as above.

FIG. 8 shows another variation of electrode assembly 160 where first and second electrodes 162, 164 are each affixed to assembly 160 via supports 170, 172, respectively. As above, common return electrode 166 may be separated by insulator 168 and fluid lumen opening 174 may be defined between electrodes 162, 164. In this variation, electrodes 162, 164 may further include an arcuate extension 176, 178, respectively, which curves circumferentially with respect to assembly 160.

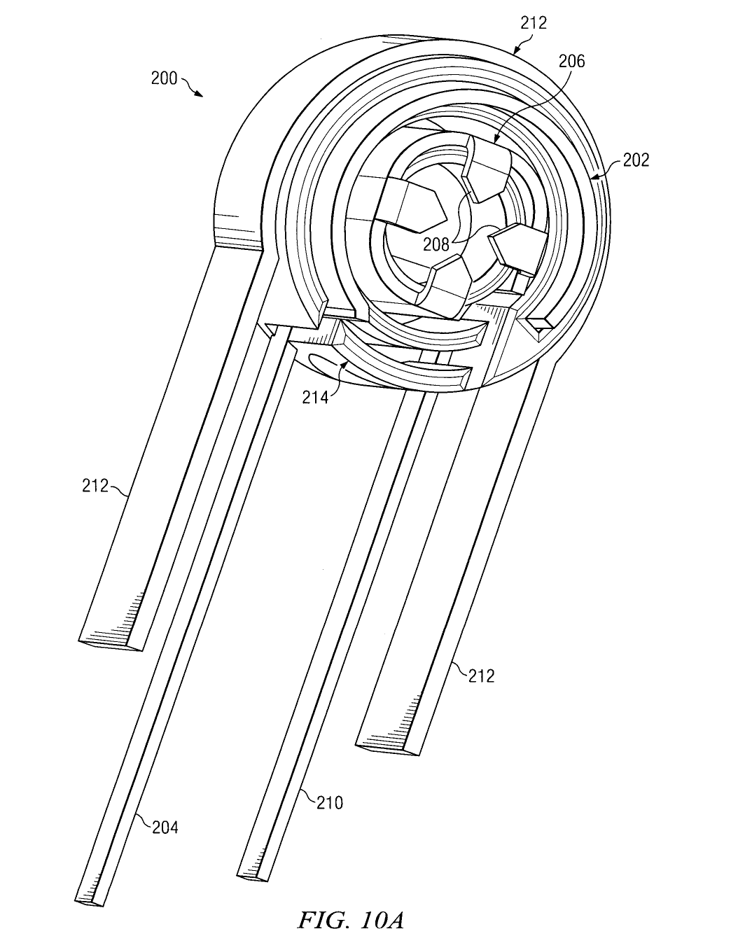

FIG. 9 shows a variation similar to that in FIG. 8 where first and second electrodes 182, 184 are each affixed to assembly 180 via supports 190, 192, respectively. Each electrode 182, 184 may similarly include an arcuate extension 196, 198 which curves circumferentially while sharing a common return electrode 186 separated by insulator 188. Lumen opening 194 may also be defined between electrodes 182, 184 for infusing saline and/or removing debris and fluids from the tissue treatment area. In this particular variation, a cross-sectional shape of the assembly 180 may define an elliptical shape where a major axis of the ellipse is in-line with the positioning of the electrodes 182, 184, as shown. As above, although an elliptical shape is shown, other variations and configurations may be utilized depending upon the desired effects and use of the device.

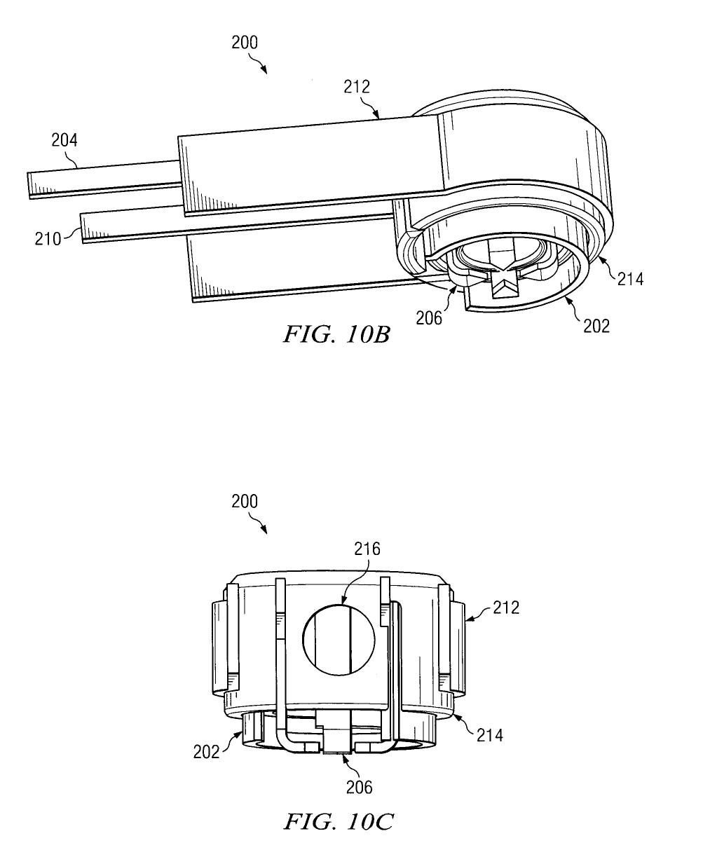

In yet another variation of a multi-electrode assembly, FIG. 10A shows a perspective view of an assembly 200 which utilizes a circumferentially-shaped first electrode 202 which at least partially surrounds a second electrode 206. First electrode 202 may be powered via cable or wire 204 and second electrode 206 may be powered via cable or wire 210 while each electrode as well as common return electrode 212 are electrically isolated from one another via insulator 214, which maintains a separation between each respective element. Second electrode 206 may further comprise one or more prongs or members 208 which project radially inward from electrode 206. Although four prongs 208 are shown evenly spaced around a circumference of second electrode 206, fewer or more prongs may be used in alternative patterns. Moreover, first electrode 202 may extend almost fully around a circumference of second electrode 206 or partially as shown.

FIG. 10B shows another perspective side view of assembly 200 illustrating first and second electrodes 202, 206 projecting from assembly 200. Additionally, FIG. 10C shows an end view of assembly 200 (with return electrode 212 partially removed for clarity) illustrating first and second electrodes 202, 206 and fluid lumen 216 defined through assembly 200 for infusing saline and/or drawing debris and fluids therethrough.

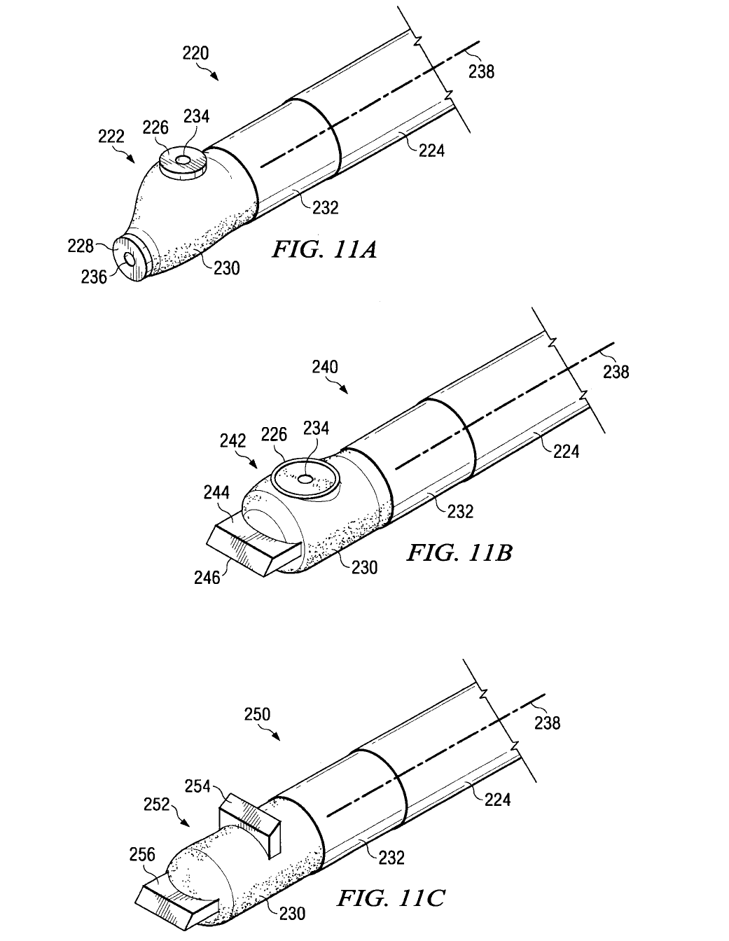

Although the multiple electrodes may positioned along a common surface and placed adjacent to one another, other examples for utilizing multiple electrodes may entail positioning the electrodes in various configurations relative to one another as well as positioning alternative types of electrodes to effect different treatments for different tissue types. One example is shown in the perspective side view of FIG. 11A which illustrates an electrosurgical instrument 220 having a multiple electrode assembly 222 disposed upon a distal end of a shaft 224, as described above. Electrode assembly 222 may utilize a first electrode 226 positioned at an angle, e.g., 90°, relative to a longitudinal axis 238 of shaft 224. A second electrode 228 may be positioned at a distal end of assembly 222 such that first and second electrodes 226, 228 are separated and angled, in this case perpendicular, relative to one another.

Although both electrodes 226, 228 are illustrated as ring-type electrodes which are configured for tissue ablation (e.g., for shaping articular cartilage or chondral defects), one or both electrodes 226, 228 may be shaped into other electrode configurations to effect other treatments, such as tissue cutting or resection. Additionally, one or both electrodes 226, 228 may include a fluid lumen 234, 236, respectively, for infusing a fluid such as saline and/or for drawing debris and fluid back into the openings. Both electrodes 226, 228 may be electrically isolated from one another as well as from common return electrode 232 by insulator 230. Such an assembly utilizing multiple electrodes in different configurations may allow the user to utilize a single device for treating different tissue regions within, e.g., a joint, where space is limited without having to withdraw and introduce multiple instruments into the tissue region.

FIG. 11B shows another variation of an instrument 240 having an electrode assembly 242 with multiple electrodes having different configurations. First electrode 226 may be a ring-type electrode for ablating tissue, as above, while second electrode 244 may be configured in this example as having a tapered or pointed edge 246, much like a chisel, for facilitating a more aggressive tissue treatment, such as cutting or resection. This assembly 242 may accordingly allow the user not only to ablate tissue regions but also cut and resect tissue with a single instrument thereby obviating the need for multiple separate instruments or for withdrawing and introducing multiple instruments.

Yet another variation is shown in the perspective view of electrode assembly 252 disposed upon instrument 250 in FIG. 11C. In this variation, first electrode 254 and second electrode 256 may be both configured with tapered edges, e.g., chisel-type configurations, so as to present cutting edges for tissue cutting or resection. Other variations for electrode configurations and combinations of various types of electrode configurations may be utilized and are intended to be included within this disclosure.

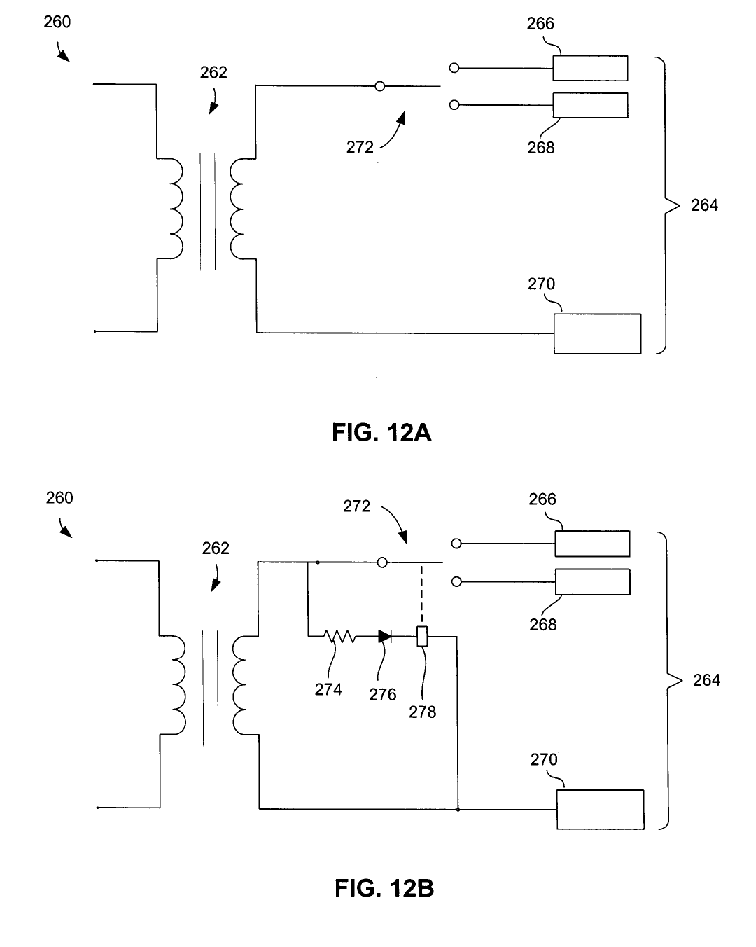

In utilizing the two or more active electrodes on a single electrosurgical instrument in any of variations described herein, a relay or switch may be used to select which of the electrodes are powered to deliver the output energy. An illustration of a relatively simple switch is shown in the schematic illustration of FIG. 12A, which shows power supply 260 transferring energy through, e.g., transformer 262, to power the electrode assembly 264. Relay 272 may switch the current from either first or second electrode 266, 268 to power the appropriate electrode and also to allow the current to flow to return electrode 270. Switch 272 may be actuated manually by the user or automatically by a controller. With each electrode 266, 268 being electrically isolated from one another and from return electrode 270, the current flowing through electrode assembly 264 is applied to the tissue to be treated, as described above.

The example in FIG. 12A or any of the schematic illustrations herein demonstrating examples for controlling and/or powering the electrode assembly may be applicable to any of the electrode configurations described herein, as practicable. The schematic representations of each electrode may be configured into any of the variations described herein or as known in the art and in any combination of different electrode types on a single instrument to effect the treatment of multiple tissue types utilizing a single electrosurgical device.

FIG. 12B shows a variation in the schematic illustration where a control coil of relay 278 may be powered via the energy output, e.g., RF energy, delivered to the electrodes. The control circuit may include some rectification so as to regulate the supplied voltage. For example, resistor 274 and diode 276 may be included so as to engage relay 278 if a voltage level above a predetermined threshold voltage is applied, thereby automatically actuating relay 278 to switch between either electrode 266, 268.

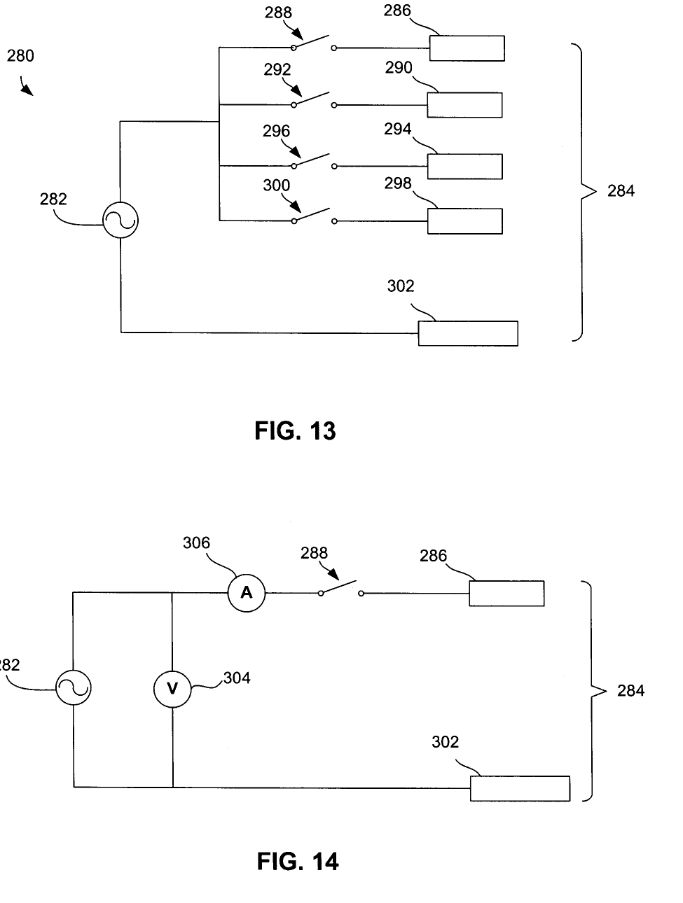

In yet additional variations where an electrode assembly has more than two electrodes, each electrically isolated electrode 286, 290, 294, 298 may each include an individually actuatable relay 288, 292, 296, 300, respectively, as illustrated in FIG. 13. As illustrated in schematic 280, this particular variation shows an example of an electrode assembly 284 having four electrodes 286, 290, 294, 298 actuatable via power source 282. The electrodes may be connected in parallel with one another and with a common return electrode 302. Each of the relays 288, 292, 296, 300 may be individually actuatable, as described above, such that the current may be applied to one, all, or any combination of the electrodes to effect the desired tissue treatment.

Each of the isolated electrodes may be designed such that each includes a voltage and/or current measurement device to measure each applied parameter. FIG. 14 illustrates an example of how a voltage meter 304 may be connected in parallel with power source 282 and/or ammeter 306 may be connected in series 306 with a particular electrode 286 to measure the applied voltage and current, respectively. Such a configuration may be applied to all or a few of the electrodes utilized. With these measured values, impedance and power loads may be calculated. Once an ablative effect has been established at one particular electrode upon the tissue being treated, the load impedance generally increases. With changes in the load impedance detected, a generator control circuitry, e.g., a microprocessor or hardware controller, may be configured to track changes in the load impedance at a given electrode and to make a determination to activate subsequent electrodes.

An example of this is determination is illustrated by the activation of electrode 286 with relay 288 contacting the circuit. As the tissue is treated, voltage meter 304 and ammeter 306 may monitor their respective signals which are used to calculate load impedance. When the load impedance reaches a predetermined threshold level, the system may be configured to then actuate relay 292 to activate electrode 290. This process may be repeated until all relays have been actuated and all electrodes are activated. Alternatively, the processor may be configured to activate subsequent electrodes based upon the measured current or the delivered power to minimize any current or power spikes initially delivered to the electrodes to facilitate the ablative effects on the tissue being treated.

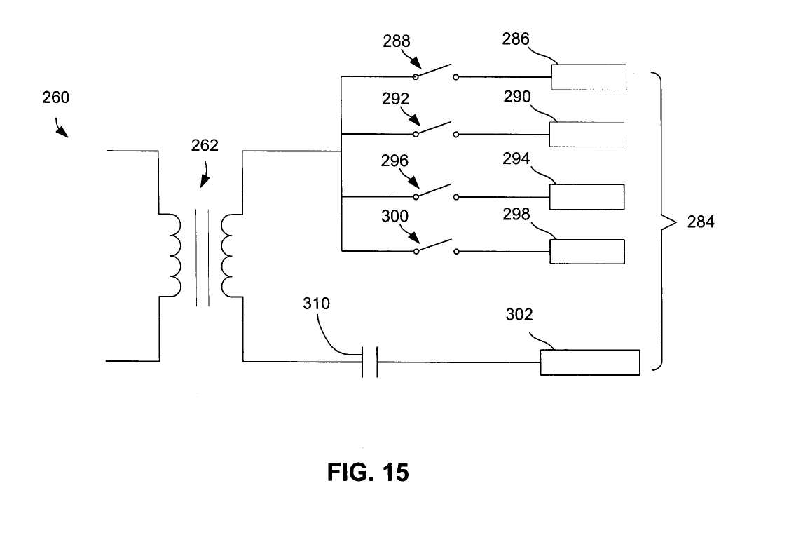

FIG. 15 illustrates another variation of an electrode assembly utilizing multiple electrodes in electrode assembly 284. In this variation, power may be transferred from power supply 260 via transformer 262 to the multiple electrode assembly 284. Return electrode 302 may further include a capacitor 310 to block undesired current signals, such as any direct-current bias which may be introduced to the tissue treatment site.

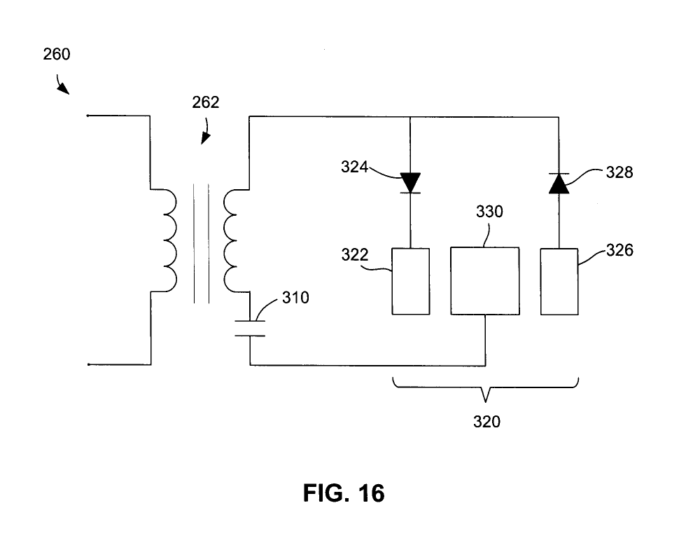

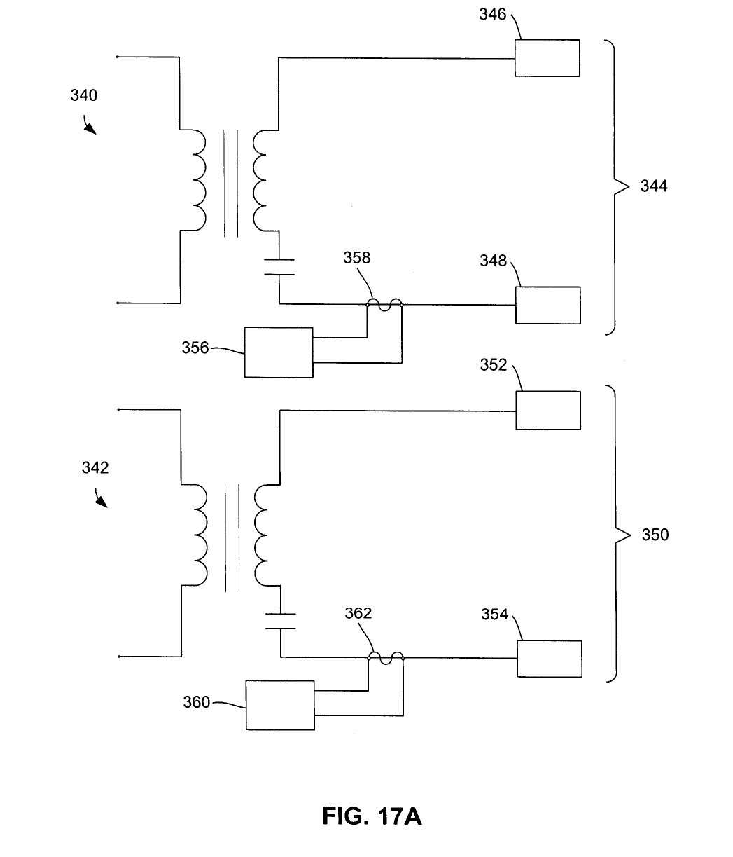

With the potential of activating multiple electrodes, one method for limiting the power that can be delivered to each electrode is shown in the schematic illustration of FIG. 16. The periodic waveform typically delivered by the power supply 260 may be utilized to deliver the power equally between the number of electrodes which may have been activated. Although the illustration of electrode assembly 320 shows a first and second electrode 322, 326 with common return electrode 330, this is merely illustrative and any number of return electrodes may be utilized as described herein. In any case, each active electrodes 322, 326 may be electrically connected to power supply 260 through respective diodes 324, 328. When the power supply is activated, the respective diodes 324, 328 may limit the activation of each electrode 322, 326 to only half of each cycle of the output waveform (or to 1/N of each cycle of the output waveform, where N is the number of active electrodes through which current is flowing). Use of the diodes may help to ensure that the power is equally shared between each active electrode independently of the load that may exist between each electrode and the return electrode.

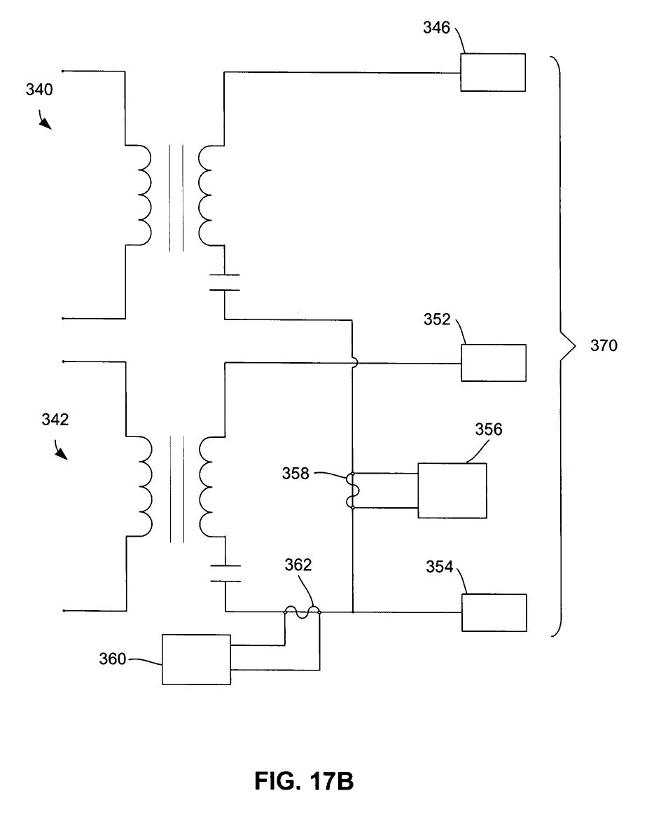

While a single power supply may be shared between multiple numbers of electrodes, another variation for delivering power to multiple electrodes is shown in FIG. 17A, which shows multiple electrodes 346, 352 powered from independent, separately controlled power supplies 340, 342. Each electrode assembly 344, 350 (shown as two electrodes 346, 352 in this variation although additional electrodes may be utilized in other variations) may include respective return electrodes 348, 354 as well as respective current monitors 356, 360 which are configured 358, 362 to monitor a current level in each electrode assembly 344, 350. Each power supply 340, 342 can be independently adjusted depending upon the measured current levels received from each electrode assembly 344, 350 to maintain a constant level of power applied by the multiple electrodes at the tissue site.

FIG. 17B shows another variation of multiple independent, separately controlled electrodes similar to that described in FIG. 17A, whereas this variation utilizes an electrode assembly 370 which similarly utilizes independently controllable power supplies 340, 342 but which utilizes a common return electrode 354 for both active electrodes 346, 352. This particular variation may be suitable for use with larger electrodes or for devices where its profile is desirably minimized.

Other modifications and variations can be made to the disclosed embodiments without departing from the subject invention. For example, other numbers and arrangements of the active electrodes and their methods for use are possible. Similarly, numerous other methods of ablating or otherwise treating tissue using electrosurgical probes will be apparent to the skilled artisan. Moreover, the instruments and methods described herein may be utilized in other regions of the body (e.g., shoulder, knee, etc.) and for other tissue treatment procedures (e.g., chondroplasty, menisectomy, etc.). Thus, while the exemplary embodiments have been described in detail, by way of example and for clarity of understanding, a variety of changes, adaptations, and modifications will be obvious to those of skill in the art. Therefore, the scope of the present invention is limited solely by the appended claims.

What is claimed is:

1. A tissue treatment system, comprising:
an electrode assembly sized for insertion within a body space and having at least a first electrode and a second electrode,
wherein the first electrode is configured to treat a first tissue type, and
wherein the second electrode is configured to treat a second tissue type which is different from the first tissue type.

2. The system of claim 1 wherein the first electrode and second electrode are interdigitated with respect to one another.

3. The system of claim 1 wherein the first electrode circumferentially surrounds the second electrode.

4. The system of claim 1 wherein the first electrode is positioned at an angle relative to the second electrode along the assembly such that the first electrode is aligned to treat a first tissue region within the body space and the second electrode is aligned to treat a second tissue region which is different from the first tissue region within the body space.

5. The system of claim 1 wherein the second electrode is configured to ablate or resect tissue.

6. The system of claim 1 wherein the first electrode and second electrode are configured to activate sequentially.

7. The system of claim 1 further comprising at least one sensor for measuring an electrical parameter of the first and/or second electrodes.

8. The system of claim 1 further comprising at least one return electrode in proximity to the first and/or second electrodes.

9. The system of claim 1 further comprising a fluid lumen defined in proximity to the first and second electrodes.

10. A method of treating tissue, comprising:
advancing an electrode assembly within a body space;
treating a first tissue type within the space with a first electrode of the assembly;
treating a second tissue type within the space with a second electrode of the assembly, wherein the first and second tissue types are different from one another.

11. The method of claim 10 wherein advancing comprises positioning the electrode assembly via an elongate shaft within the body space.

12. The method of claim 10 wherein advancing comprises positioning the electrode assembly within a joint space of a patient body.

13. The method of claim 10 further comprising flooding the body space with saline prior to treating a first tissue type.

14. The method of claim 10 wherein treating a first tissue type comprises treating a region of cartilage tissue.

15. The method of claim 14 wherein treating a second tissue type comprises treating a region of meniscus tissue.

16. The method of claim 10 wherein treating a first tissue type comprises ablating the first tissue type.

17. The method of claim 10 wherein treating the second tissue type is treated after treating the first tissue type.

18. A method of controlling multiple electrodes, comprising:
applying energy to a first electrode for treating one or more tissue regions within a body space while measuring an electrical parameter of the first electrode;
if the electrical parameter reaches a predetermined value, applying energy to a second electrode for treating the tissue region within the body space.

19. The method of claim 18 further comprising positioning an electrode assembly upon which the first and second electrodes are disposed via an elongate shaft within the body space prior to applying energy to a first electrode.

20. The method of claim 18 further comprising flooding the body space with saline prior to applying energy to a first electrode.

21. The method of claim 18 wherein applying energy to a first electrode comprises treating a region of cartilage tissue.

22. The method of claim 18 wherein measuring an electrical parameter further comprises calculating an impedance load of the tissue region.

23. The method of claim 18 wherein applying energy to a second electrode comprises simultaneously applying energy to the first and second electrodes.

24. The method of claim 18 further comprising applying energy to at least one additional electrode if an electrical parameter of the second electrode reaches the predetermined value.

25. The system of claim 1 further comprising a power supply and said first and second electrodes being independently electrically connected to said power supply.

26. The method of claim 10 further comprising independently activating one of the first electrode and the second electrode.
